(19) United States
(12) Patent Application Publication
(10) Pub. No.: US 2011/0034970 A1
(43) Pub. Date: Feb. 10, 2011

(54) NEUROSTIMULATION LEAD AND SYSTEM AND METHODS OF MAKING AND USING

(75) Inventor: John Michael Barker, Ventura, CA (US)

Correspondence Address: Boston Scientific Neuromodulation Corp.;c/o Frommer Lawrence & Haug LLP, 745 Fifth Ave, NEW YORK, NY, 10151, US

(73) Assignee: Boston Scientific Neuromodulation Corporation, Valencia, CA (US)

(21) Appl. No.: 12/535,460

(22) Filed: Aug. 4, 2009

(51) Int. Cl. A61N 1/36; A61N 1/05

(52) U.S. Cl. 607/72; 607/116

(57) ABSTRACT

A lead includes an outer tube body, an inner tube body, conductors, and electrodes. A portion of the inner tube body may be disposed in the outer tube body lumen. The conductors are optionally partially disposed within the inner tube body lumen, wherein a distal end of each conductor extends beyond a distal end of the inner tube body. Each electrode is optionally coupled to a conductor. The outer tube body may be slideable over the inner tube body between a first position in which the conductors and electrodes are disposed in the outer tube body lumen and a second position in which the outer tube body is partially retracted to expose the conductors and electrodes. The lead is optionally configured and arranged such that at least a portion of the inner tube body remains disposed in the outer tube body lumen after completion of implantation of the lead.

### FIELD

The present invention is directed to the area of implantable electrical stimulation systems and methods of making and using the systems. The present invention is also directed to implantable electrical stimulation leads having an outer tube body that is slideable over an inner tube body to dispose the electrodes within a lumen of the outer tube body, as well as methods of making and using the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more lead bodies, and an array of stimulator electrodes coupled to each lead body. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead includes an outer tube body defining a lumen extending through the outer tube body; an inner tube body defining a lumen extending through the inner tube body; a plurality of conductors; and a plurality of electrodes. In some embodiments, at least a portion of the inner tube body is disposed in the lumen of the outer tube body. In some embodiments, the plurality of conductors are partially disposed within the lumen of the inner tube body, wherein a distal end of each of the conductors extends beyond a distal end of the inner tube body. In at least some embodiments, each electrode is coupled to one of the conductors. In some embodiments, the outer tube body is slideable over the inner tube body between a first position in which the conductors and the electrodes are disposed in the lumen of the outer tube body and a second position in which the outer tube body is partially retracted to expose the conductors and electrodes. In some embodiments, the lead is configured and arranged such that at least a portion of the inner tube body remains disposed in the lumen of the outer tube body after completion of implantation of the lead in a patient.

In one embodiment, a stimulation system includes an implantable pulse generator and a lead coupled to the implantable pulse generator. In some embodiments, the lead includes an outer tube body defining a lumen extending through the outer tube body, an inner tube body defining a lumen extending through the inner tube body, a plurality of conductors and a plurality of electrodes. In some embodiments, at least a portion of the inner tube body is disposed in the lumen of the outer tube body. In some embodiments, a distal end of each of the conductors extends beyond a distal end of the inner tube body. In some embodiments, each electrode is coupled to one of the conductors. In some embodiments, the outer tube body is slideable over the inner tube body between a first position in which the conductors and the electrodes are disposed in the lumen of the outer tube body and a second position in which the outer tube body is partially retracted to expose the conductors and electrodes. In some embodiments, the lead is configured and arranged such that at least a portion of the inner tube body remains disposed in the lumen of the outer tube body after completion of implantation of the lead in a patient.

In one embodiment, a method of implanting a lead includes implanting a lead into a patient wherein the lead includes an outer tube body defining a lumen extending through the outer tube body; an inner tube body defining a lumen extending through the inner tube body; a plurality of conductors; and a plurality of electrodes. In some embodiments, at least a portion of the inner tube body is disposed in the lumen of the outer tube body. In some embodiments, the plurality of conductors are partially disposed within the lumen of the inner tube body. In some embodiments, a distal end of the conductors extends beyond a distal end of the inner tube body. In some embodiments, each electrode is coupled to one of the conductors. In some embodiments, a method of implanting a lead includes disposing the outer tube body in a first position in which each of the plurality of conductors and each of the plurality of electrodes are disposed in the lumen of the outer tube body. In some embodiments, a method of implanting a lead includes partially retracting the outer tube body to expose at least one electrode coupled to at least one conductor.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic perspective view of one embodiment of an electrical stimulation system, according to the invention;

FIG. 2 is a schematic perspective view of one embodiment of a proximal portion of a lead and a control module of an electrical stimulation system, according to the invention;

FIG. 3 is a schematic perspective view of one embodiment of a proximal portion of a lead, a lead extension, and a control module of an electrical stimulation system, according to the invention;

FIG. 4 is a schematic side view of one embodiment of a distal portion of a lead with the outer tube body extended such that the electrodes are disposed within a lumen of the outer tube body, according to the invention;

FIG. 5 is a close-up schematic view of the distal portion of the lead of FIG. 4;

FIG. 6 is a cross-sectional view of the distal portion of the lead of FIG. 5 at line 6-6;

FIG. 7 is a cross-sectional view of the distal portion of the lead of FIG. 5 at line 7-7;

FIG. 8 is a schematic perspective view of one embodiment of a distal portion of a lead with the outer tube body partially retracted, according to the invention;

FIG. 9A is a schematic perspective view of one embodiment of a distal portion of a lead with the outer body extended such that the electrodes are disposed within a lumen of the outer tube body, according to the invention;

FIG. 9B is a schematic perspective view of one embodiment of a distal portion of a lead with the outer tube body partially retracted, according to the invention; and

FIG. 10 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation leads having an outer tube body that is slideable over an inner tube body to dispose the electrodes within a lumen of the outer tube body, as well as methods of making and using the leads and electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Examples of electrical stimulation systems with leads are found in, for example, U.S. Pat. Nos. 6,181,969; 6,516,227; 6,609,029; 6,609,032; and 6,741,892; and U.S. patent application Ser. Nos. 10/353,101, 10/503,281, 11/238,240; 11/319,291; 11/327,880; 11/375,638; 11/393,991; and 11/396,309, all of which are incorporated by reference.

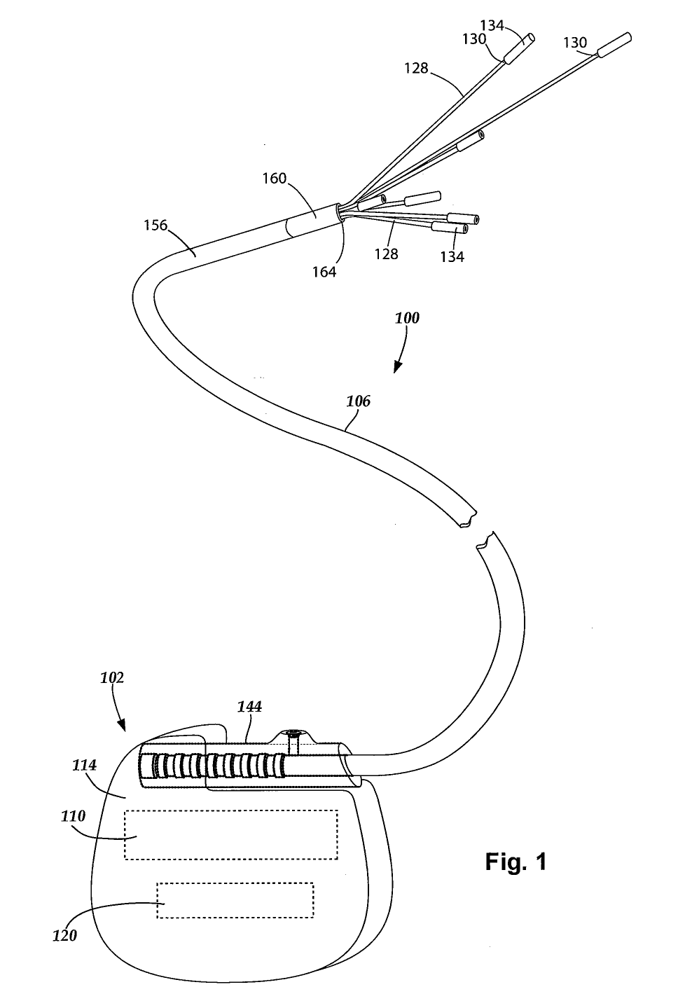

FIG. 1 illustrates schematically one embodiment of an electrical stimulation system 100. The electrical stimulation system includes a control module (e.g., a stimulator or pulse generator) 102, a plurality of electrodes 134, and at least one lead body 106 coupling the plurality of electrodes 134 to the control module 102. The plurality of electrodes 134 are electrically coupled to the control module 102 via conductors 128. Each electrode 134 is coupled to a distal end 130 of a conductor 128.

The electrical stimulation system schematically illustrated in FIG. 1 also includes an inner tube body 160 and an outer tube body 156, which are described in more detail below.

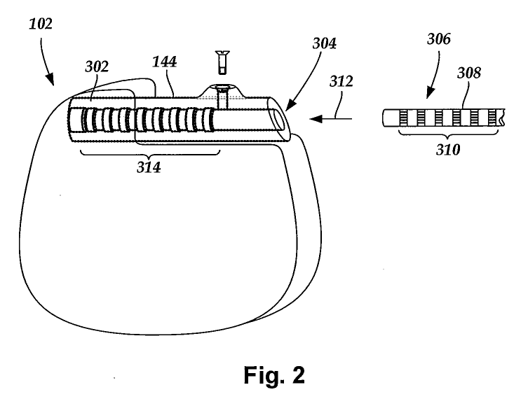

The control module 102 typically includes an electronic subassembly 110 and an optional power source 120 disposed in a sealed housing 114. The control module 102 typically includes a connector 144 (FIGS. 1 and 2; see also 350 of FIG. 3) into which the proximal end of the one or more lead bodies 106 can be plugged to make an electrical connection via conductive contacts on the control module 102 and terminals (e.g., 310 in FIG. 2 and 336 in FIG. 3) on each of the one or more lead bodies 106.

It will be understood that the electrical stimulation system can include more, fewer, or different components and can have a variety of different configurations including those configurations disclosed in the electrical stimulation system references cited herein. For example, one or more lead extensions 324 (see FIG. 3) can be disposed between the one or more lead bodies 106 and the control module 102 to extend the distance between the one or more lead bodies 106 and the control module 102 of the embodiment shown in FIG. 1.

Terminals (e.g., 310 in FIG. 2 and 336 of FIG. 3) are typically disposed at the proximal end of the one or more lead bodies 106 for connection to corresponding conductive contacts (e.g., 314 in FIG. 2 and 340 in FIG. 3) in connectors (e.g., 144 in FIGS. 1 and 2; 322 and 350 of FIG. 3) disposed on, for example, the control module 102 (or to other devices, such as conductive contacts on a lead extension, an operating room cable, or an adaptor). Conductors 128 extend from the terminals (e.g., 310 in FIG. 2 and 336 in FIG. 3) to the electrodes 134. Typically, one or more electrodes 134 are electrically coupled to a terminal (e.g., 310 in FIG. 2 and 336 in FIG. 3) via one or more conductors 128.

In at least some embodiments, leads are coupled to connectors disposed on control modules. In FIG. 2, a lead 308 is shown configured and arranged for insertion into the control module 102. The connector 144 includes a connector housing 302. The connector housing 302 defines at least one port 304 into which a proximal end 306 of a lead 308 with terminals 310 can be inserted, as shown by directional arrow 312. The connector housing 302 also includes a plurality of conductive contacts 314 for each port 304. When the lead 308 is inserted into the port 304, the conductive contacts 314 can be aligned with the terminals 310 on the lead 308 to electrically couple the control module 102 to the electrodes (134 of FIG. 1) disposed at a distal end of the lead 308. Examples of connectors in control modules are found in, for example, U.S. Pat. No. 7,244,150 and U.S. patent application Ser. No. 11/532,844, which are incorporated by reference.

In FIG. 3, a connector 322 is disposed on a lead extension 324. The connector 322 is shown disposed at a distal end 326 of the lead extension 324. The connector 322 includes a connector housing 328. The connector housing 328 defines at least one port 330 into which a proximal end 332 of a lead 334 with terminals 336 can be inserted, as shown by directional arrow 338. The connector housing 328 also includes a plurality of conductive contacts 340. When the lead 334 is inserted into the port 330, the conductive contacts 340 disposed in the connector housing 328 can be aligned with the terminals 336 on the lead 334 to electrically couple the lead extension 324 to the electrodes (134 of FIG. 1) disposed at a distal end (not shown) of the lead 334.

In at least some embodiments, the proximal end of a lead extension is similarly configured and arranged as a proximal end of a lead. The lead extension 324 may include a plurality of conductive wires (not shown) that electrically couple the conductive contacts 340 to a proximal end 348 of the lead extension 324 that is opposite to the distal end 326. In at least some embodiments, the conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a connector disposed in another lead extension. In other embodiments, the proximal end 348 of the lead extension 324 is configured and arranged for insertion into a connector disposed in a control module. As an example, in FIG. 3 the proximal end 348 of the lead extension 324 is inserted into a connector 350 disposed in a control module 352.

Returning to FIG. 1, the outer tube body 156 defines a lumen 158 (see FIG. 6) extending through the outer tube body 156. The inner tube body 160 defines a lumen 162 (see FIG. 7) extending through the inner tube body 160. At least a portion of the inner tube body 160 is disposed in the lumen 158 of the outer tube body 156.

In some embodiments, the inner tube body 160, the outer tube body 156, or both the inner tube body 160 and the outer tube body 156 are made of a non-conductive, biocompatible material such as, for example, silicone, polyurethane, polyetheretherketone (“PEEK”), and the like or combinations thereof. In some embodiments, the inner tube body 160, the outer tube body 156, or both the inner tube body 160 and the outer tube body 156 are made of a conductive, biocompatible material. For example, the inner tube body 160 or the outer tube body 156 or both can optionally be made of a thin walled hypodermic needle tubing. The inner tube body 160 and the outer tube body 156 can be made by any process known to those of skill in the art including, for example, molding or extruding. Preferably, the inner tube body 160 and the outer tube body 156 are flexible.

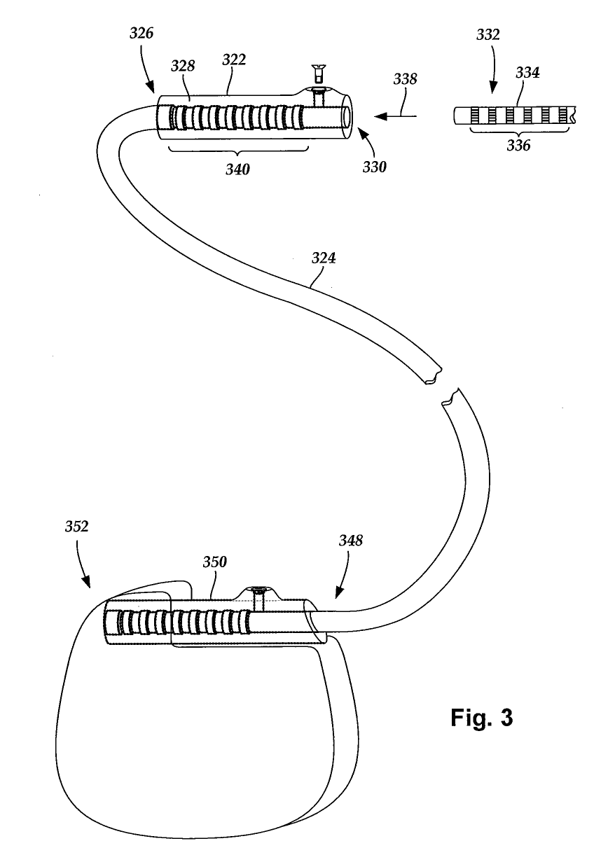

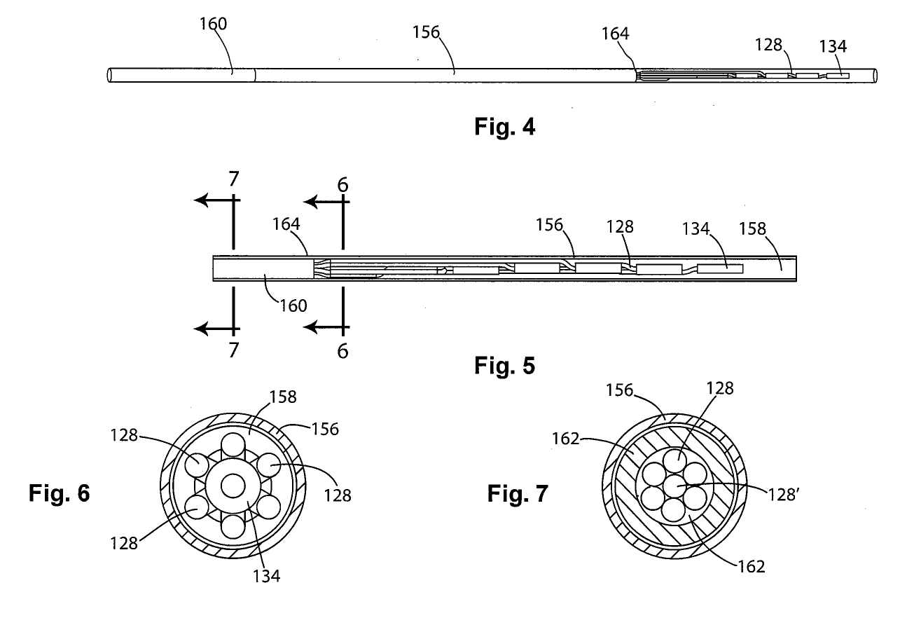

The outer tube body 156 is slideable over the inner tube body 160 and may be disposed in a variety of positions relative to the inner tube body 160. For example, in a first position, the outer tube body 156 extends distally over the inner tube body 160 as illustrated schematically in FIGS. 4 and 5. In this first position, the outer tube body 156 extends distally over the inner tube body 160 such that at least one electrode 134 is disposed within the lumen 158 of the outer tube body 156. In at least some embodiments, all of the electrodes 134 are disposed within the lumen 158 of the outer tube body 156 when the outer tube body 156 is in a first position, extended distally over the inner tube body 160.

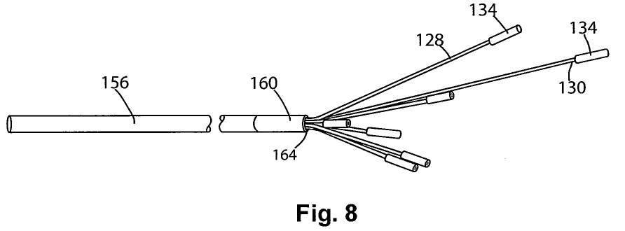

In a second position, the outer tube body 156 is at least partially retracted to expose at least one electrode 134 as illustrated schematically in FIGS. 1 and 8. In at least some embodiments, when the outer tube body 156 is at least partially retracted, all of the electrodes 134 are exposed.

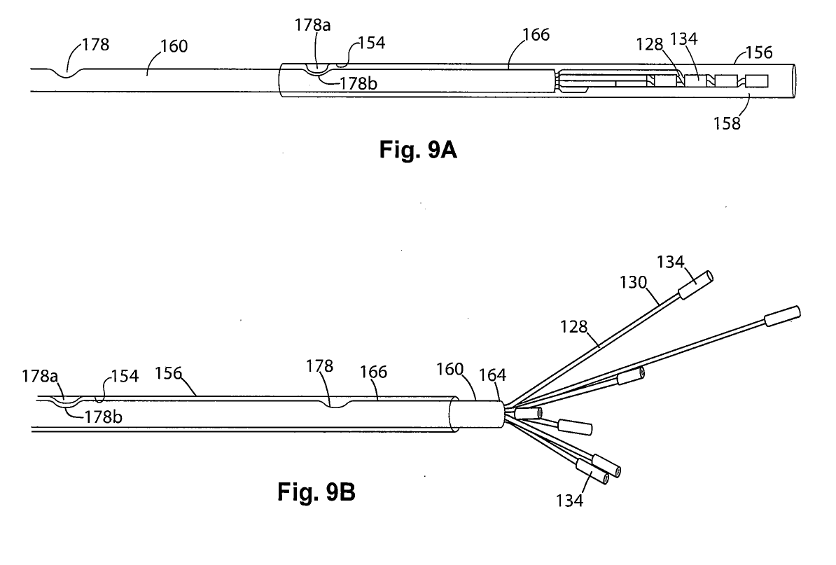

In some embodiments, the outer tube body 156 includes at least one locking member 178 (see FIGS. 9A and 9B). The locking member 178 may be located anywhere on the outer tube body 156. In some embodiments, the locking member 178 is disposed on a proximal portion of the outer tube body 156. In some embodiments, the locking member 178 is disposed on the exterior surface of the outer tube body 156. In other embodiments, the locking member 178 is disposed on an interior surface 154 (see FIGS. 9A and 9B) of the outer tube body 156.

In some embodiments, the inner tube body 160 comprises at least one locking member 178 (FIGS. 9A and 9B). The locking member 178 may be located anywhere on the inner tube body 160. In some embodiments, the locking member 178 is disposed at a proximal portion of the inner tube body 160. In some embodiments, the locking member 178 is disposed on the exterior surface 166 of the inner tube body 160 (see FIGS. 9A and 9B).

In still other embodiments, the inner tube body 160, the outer tube body 156 or both the inner tube body 160 and the outer tube body 156 comprise at least one locking member 178 (see FIGS. 9A and 9B). For example, the inner tube body 160, the outer tube body 156, or both the inner tube body 160 and the outer tube body 156 may comprise two or more locking members 178. In some embodiments, the inner tube body 160, the outer tube body 156 or both include a first locking member 178 to maintain the outer tube body 156 in a first position and a second locking member 178 to maintain the outer tube body 156 in a second position.

The locking member 178 is configured and arranged to maintain a position of the outer tube body 156. For example, the locking member 178 may be configured and arranged to maintain an axial position of the outer tube body 156 with respect to the position of the inner tube body 160. In some embodiments, the locking member 178 is configured and arranged to maintain the outer tube body 156 in a first position in which the conductors 128 and the electrodes 134 are disposed in the lumen 158 of the outer tube body 156 (see FIG. 9A). Alternatively or additionally, the locking member 178 may be configured and arranged to maintain the outer tube body 156 in a second position in which the outer tube body 156 is partially retracted to expose at least one electrode 134 (see FIG. 9B).

The locking member 178 may have any shape including, for example, a protrusion or a depression. In some embodiments, a locking member 178 disposed on the outer tube body 156 comprises a protrusion 178a extending from the interior surface 154 of the outer tube body 156 into the lumen 158 of the outer tube body 156 as illustrated schematically in FIGS. 9A and 9B.

In some embodiments, a locking member 178 disposed on the inner tube body 160 comprises a depression 178b in the exterior surface 166 of the inner tube body as illustrated schematically in FIGS. 9A and 9B. As will be recognized, locking members 178, such as locking members 178a and 178b, may function cooperatively to maintain a position, such as an axial position, of the outer tube body 156 with respect to the position of the inner tube body 160. In some embodiments, the locking member 178 is a detent. For example, the locking member 178 can optionally be a non-permanent (reversible) detent.

Returning to FIG. 1, the electrodes 134 can be formed using any conductive, biocompatible material. Examples of suitable materials include metals, alloys (e.g., 90% platinum/10% iridium), conductive polymers, conductive carbon, and the like, as well as combinations thereof. The number of electrodes 134 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used.

The electrodes 134 may have any shape. For example, the electrodes 134 may have a shape in the form of a cylinder, sphere, cube, parallelepiped, or any other regular or irregular shape. In at least some embodiments, the electrodes 134 have a cylindrical shape as illustrated schematically in FIG. 1. Each electrode 134 is electrically coupled to at least one conductor 128. In at least some embodiments, each electrode 134 is electrically coupled to only one conductor 128. For example, one end of an electrode 134 may be coupled to a distal end 130 of a conductor 128 as illustrated schematically in FIG. 1. The electrode 134 may be coupled to the conductor 128 in any manner including, for example, by welding or crimping. In at least some embodiments, only one end of the electrode 134 is coupled to the conductor 128 and the remainder of the electrode 134 is left exposed. This allows the remainder of the electrode 134 to contact the tissue to be stimulated.

As described above, the electrodes 134 are electrically coupled to the control module 102 via one or more conductors 128. In at least some embodiments, the number of conductors 128 is equal to the number of electrodes 134. In other embodiments, two or more electrodes 134 may be coupled to one of the conductors 128.

The conductors 128 may be made of any conductive, biocompatible material including, for example, metals, alloys and the like. Each conductor 128 preferably extends from a terminal (e.g., 310 in FIG. 2) at a proximal end of the lead to an electrode 134 at a distal end of the lead. At least a portion of each conductor 128 is disposed within the lumen 162 of the inner tube body 160. Preferably, each conductor 128 is disposed within the entire length of the lumen 162 of the inner tube body 160.

A distal end 130 of at least one conductor 128 extends beyond a distal end 164 of the inner tube body 160. In some embodiments, the conductors 128 have different lengths such that the electrodes 134 are disposed in a staggered and non-overlapping position within the lumen 158 of the outer tube body 156 when the outer tube body 156 is extended distally over the inner tube body 160 and is disposed in a first position (see FIGS. 4 and 5).

At least the portion of the conductor 128 that is disposed in the lumen 162 of the inner tube body 160 and the portion of the conductor 128 that extends beyond a distal end 164 of the inner tube body 160 are coated with, or otherwise disposed within, a non-conductive material such that these portions of the conductor 128 are insulated (electrically isolated). Preferably, the entire length of the conductor 128 is insulated except for the distal tip of the conductor 128, which is coupled to the electrode 134, and the proximal tip of the conductor 128, which is coupled to a terminal 310 (see, e.g., FIG. 2).

Conductors 128 disposed in the lumen 162 of the inner tube body 160 may be arranged in a circular array as illustrated schematically in FIGS. 6 and 7. The circular array of conductors 128 may include a centrally located conductor 128′ as illustrated schematically in FIG. 7. In other embodiments, there is no centrally located conductor 128′. In some embodiments, conductors 128 disposed in a lumen of the inner tube body 160 are arranged in a flat array. For example, conductors 128 may optionally be arranged in a lumen of the inner tube body 160 in a flat array like a ribbon cable. In at least some embodiments, the conductors 128 are flexible.

The electrical stimulation system or at least some components of the electrical stimulation system, such as the lead, are typically implanted into the body of a patient. The electrical stimulation system can be used for a variety of applications including, but not limited to, brain stimulation, neural stimulation, spinal cord stimulation, muscle stimulation, and the like.

In some embodiments, the lead is configured and arranged to be implanted through a cannula. In other embodiments, the lead is configured and arranged to be implanted through a hypodermic needle (e.g., a 20G hypodermic needle). A lead configured and arranged to be implanted through a cannula or a hypodermic needle can advantageously be implanted without the need for an invasive surgical procedure.

In some embodiments, a method of implanting a lead comprises implanting the lead into a patient wherein the outer tube body 156 of the lead is disposed in a first position in which each of the conductors 128 and each of the electrodes 134 are disposed in the lumen 158 of the outer tube body (see FIGS. 4 and 5). As described above, the lead may be implanted using a cannula, a hypodermic needle (e.g., a 20G hypodermic needle) or the like.

For example, the lead may be disposed in a hypodermic needle. The hypodermic needle can then be inserted into the patient adjacent to the tissue to be stimulated. The lead can be inserted from the hypodermic needle into the tissue of the patient. Next, the hypodermic needle may be withdrawn from the patient, leaving the lead, including the outer tube body 156, implanted in the patient. At least a portion of the inner tube body 160 remains disposed in the lumen 158 of the outer tube body 156 when the lead is implanted in a patient.

As described above, the outer tube body 156 or the inner tube body 160 or both may optionally include a locking member 178 (see FIG. 9A) that maintains the outer tube body 156 in a first position, extended distally over the inner tube body 160. In some embodiments, a method of implanting a lead comprises engaging the locking member 178 to maintain an axial position of the outer tube body 156 in a first position with respect to the position of the inner tube body 160 (see FIG. 9A) before the lead is implanted into the patient. Engaging the locking member 178 to maintain the outer tube body 156 in a first position ensures that the electrodes 134 remain disposed within the lumen 158 of the outer tube body 156 while the lead is being implanted into the patient.

Once the lead is implanted in the desired position within the patient, the outer tube body 156 can be at least partially retracted to expose at least one electrode 134 (see FIGS. 1 and 8). Preferably, after the lead is implanted in the desired position, the outer tube body 156 is at least partially retracted to expose all of the electrodes 134.

After the lead has been implanted and the outer tube body 156 has been at least partially retracted, one or more locking members 178 disposed on the outer tube body 156 or the inner tube body 160 or both may optionally be engaged. Engaging the locking member 178 maintains an axial position of the outer tube body 156 with respect to the inner tube body 160 to ensure that at least one electrode 134 remains exposed. For example, engaging the locking member 178 can maintain the outer tube body 156 in a second position with the outer tube body 156 at least partially retracted (see FIG. 9B).

After the lead has been implanted, each of the electrodes 134 can optionally be positioned with respect to the tissue to be stimulated. The electrodes 134 can optionally individually be positioned such that each electrode 134 is positioned separately from the other electrodes 134 of the lead. For example, in some embodiments, each of the electrodes 134 is positioned after the outer tube body 156 is retracted to be positioned in a second position to expose one or all of the electrodes 134. A practitioner may move the electrodes to desired positions after implantation and retraction of the outer tube body 156. In other embodiments, each of the electrodes 134 is positioned after the outer tube body 156 is retracted to be positioned in a second position and after at least one locking member 178 has been engaged to maintain the outer tube body 156 in a second position.

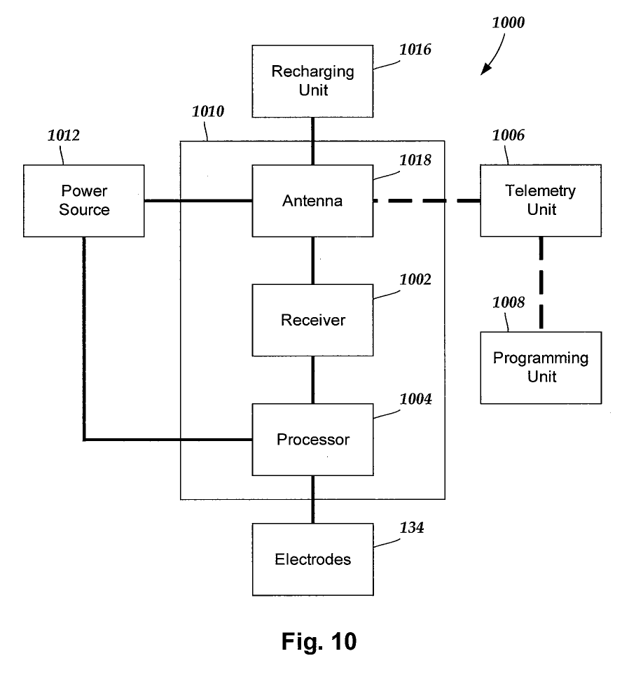

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1508. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead comprising:
an outer tube body defining a lumen extending through the outer tube body;
an inner tube body defining a lumen extending through the inner tube body, wherein at least a portion of the inner tube body is disposed in the lumen of the outer tube body;
a plurality of conductors partially disposed within the lumen of the inner tube body, wherein a distal end of each of the conductors extends beyond a distal end of the inner tube body; and
a plurality of electrodes, wherein each electrode is coupled to a one of the conductors,
wherein the outer tube body is slideable over the inner tube body between a first position in which the conductors and the electrodes are disposed in the lumen of the outer tube body and a second position in which the outer tube body is partially retracted to expose the conductors and electrodes,
and wherein the lead is configured and arranged such that at least a portion of the inner tube body remains disposed in the lumen of the outer tube body after completion of implantation of the lead in a patient.

2. The lead of claim 1, wherein the plurality of conductors are arranged in a circular array within the lumen of the inner tube body.

3. The lead of claim 1, wherein the plurality of conductors are electrically insulated from each other.

4. The lead of claim 1, wherein each of the plurality of conductors extend substantially along the length of the lumen of the inner tube body.

5. The lead of claim 1, wherein the inner tube body has no openings except for the openings for the lumen.

6. The lead of claim 1, wherein at least one electrode is cylindrical in shape.

7. The lead of claim 1, wherein the plurality of conductors have different lengths such that the electrodes are disposed in a staggered and non-overlapping position within the lumen of the outer tube body when the outer tube body is disposed in the first position.

8. The lead of claim 1, wherein the outer tube body comprises at least one locking member, wherein the locking member is configured and arranged to maintain the outer tube body in either the first position or the second position.

9. The lead of claim 1, wherein the lead is configured and arranged to be implanted through a hypodermic needle.

10. The lead of claim 8, wherein the inner tube body comprises at least one locking member and wherein the locking members cooperate to maintain an axial position of the outer tube body.

11. A stimulation system comprising:
an implantable pulse generator; and
a lead coupled to the implantable pulse generator, wherein the lead comprises:
an outer tube body defining a lumen extending through the outer tube body;
an inner tube body defining a lumen extending through the inner tube body, wherein at least a portion of the inner tube body is disposed in the lumen of the outer tube body;
a plurality of conductors partially disposed within the lumen of the inner tube body, wherein a distal end of each of the conductors extends beyond a distal end of the inner tube body; and
a plurality of electrodes, wherein each electrode is coupled to a one of the conductors,
wherein the outer tube body is slideable over the inner tube body between a first position in which the conductors and the electrodes are disposed in the lumen of the outer tube body and a second position in which the outer tube body is partially retracted to expose the conductors and electrodes,
and wherein the lead is configured and arranged such that at least a portion of the inner tube body remains disposed in the lumen of the outer tube body after completion of implantation of the lead in a patient.

12. The stimulation system of claim 11, wherein the plurality of electrodes are cylindrical in shape.

13. The stimulation system of claim 11, wherein the plurality of conductors have different lengths such that the electrodes are disposed in a staggered and non-overlapping position within the lumen of the outer tube body when the outer tube body is disposed in the first position.

14. The stimulation system of claim 11, wherein the outer tube body comprises at least one locking member, wherein the locking member is configured and arranged to maintain the outer tube body in either the first position or the second position.

15. The stimulation system of claim 11, wherein both the outer tube body and the inner tube body comprise at least one locking member, and wherein the locking members cooperate to maintain the outer tube body in either the first position or the second position.

16. A method of implanting a lead, comprising:
implanting a lead into a patient, wherein the lead comprises an outer tube body defining a lumen extending through the outer tube body; an inner tube body defining a lumen extending through the inner tube body, wherein at least a portion of the inner tube body is disposed in the lumen of the outer tube body; a plurality of conductors partially disposed within the lumen of the inner tube body, wherein a distal end of each of the conductors extends beyond a distal end of the inner tube body; and a plurality of electrodes, wherein each electrode is coupled to a one of the conductors, wherein the outer tube body is disposed in a first position in which each of the plurality of conductors and each of the plurality of electrodes are disposed in the lumen of the outer tube body; and
partially retracting the outer tube body to expose at least one electrode coupled to at least one conductor.

17. The method of claim 16, wherein implanting the lead comprises implanting the lead into the patient through a hypodermic needle.

18. The method of claim 17, comprising disposing the lead in the hypodermic needle, inserting the hypodermic needle into the patient adjacent to a tissue to be stimulated, implanting the lead from the hypodermic needle into the tissue of the patient, and then withdrawing the hypodermic needle from the patient.

19. The method of claim 16, wherein the outer tube body comprises at least one locking member and wherein before the lead is implanted into the patient, the locking member is engaged to maintain an axial position of the outer tube body with respect to the inner tube body such that the plurality of electrodes remain disposed within the lumen of the outer tube body during implantation of the lead.

20. The method of claim 16, wherein the outer tube body comprises at least one locking member and wherein after the outer tube body is retracted to expose at least one electrode, the locking member is engaged to maintain an axial position of the outer tube body with respect to the inner tube body.
